(19) United States
(12) Patent Application Publication
(10) Pub. No.: US 2021/0361537 A1
(43) Pub. Date: Nov. 25, 2021

(54) SPHERIFICATED DENTAL MEDICAMENT COMPOSITIONS

(71) Applicants: Mihran Papazian, Yerevan (AM); David Becker, Burbank, CA (US)

(72) Inventors: Mihran Papazian, Yerevan (AM); David Becker, Burbank, CA (US)

(21) Appl. No.: 16/959,144

(22) PCT Filed: Dec. 28, 2018
(86) PCT No.: PCT/US2018/067887

§ 371 Date: Jun. 29, 2020

Related U.S. Application Data
(60) Provisional application No. 62/612,462, filed on Dec. 31, 2017.

(51) Int. Cl. A61K 8/02; A61K 8/11; A61K 8/73; A61K 8/49; A61K 8/34; A61K 8/21; A61Q 11/00

(52) U.S. Cl. CPC A61K 8/025; A61K 8/11; A61K 8/733; A61K 8/4926; A61K 2800/614; A61K 8/21; A61Q 11/00; A61K 2800/56; A61K 8/347

(57) ABSTRACT

A spherificated dental medicament that includes an outer membrane containing calcium alginate and an internal core containing at least one of a mouth rinse, a mouthwash, a dental paste, and a dental gel, A method of making the spherificated dental medicament includes immersing a dental medicament composition into a solution containing either a soluble alginate or calcium salt, the dental medicament composition comprising a calcium salt or a soluble alginate and at least one of a mouth rinse, a mouthwash, a dental paste and a dental gel, and then removing the dental medicament composition from the solution following formation of a membrane around the dental medicament composition.

### CROSS-REFERENCE TO RELATED APPLICATION

This application claims the benefit of and priority to U.S. Provisional Application No. 62/612,462, filed on Dec. 31, 2017, the content of which is incorporated by reference herein in its entirety.

### FIELD OF THE INVENTION

The field of the invention relates to spherificated medicaments, specifically, spherificated dental medicaments.

### BACKGROUND OF THE INVENTION

Dental medicaments, such as toothpaste and mouthwashes, are often sold in relatively large volumes. This requires the user to measure or meter the amount of medicament that is administered orally, which introduces user error. An excessive amount of medicament may be used resulting in waste or too little medicament may be administered resulting in inadequate treatment. Thus, there is a need for improved forms and delivery methods for dental medicaments.

### SUMMARY OF THE INVENTION

In one aspect of the invention, a spherificated dental medicament comprises an outer membrane comprising calcium alginate; and an internal core containing at least one of a mouth rinse, a mouthwash, a dental paste, and a dental gel.

In another aspect of the invention, a method of making a spherificated dental medicament comprises immersing a dental medicament composition into a solution containing a calcium salt, the dental medicament composition comprising a soluble alginate and at least one of a mouth rinse, a mouthwash, a dental paste and a dental gel; and removing the dental medicament composition from the solution following formation of a membrane around the dental medicament composition, the membrane comprising calcium alginate.

In yet another aspect of the invention, a method of making a spherificated dental medicament comprises immersing a dental medicament composition into a solution containing a soluble alginate, the dental medicament composition comprising a calcium salt and at least one of a mouth rinse, a mouthwash, a dental paste and a dental gel; and removing the dental medicament composition from the solution following formation of a membrane around the dental medicament composition, the membrane comprising calcium alginate.

These and other aspects of the present invention will be apparent in view of the following description.

### BRIEF DESCRIPTION OF THE FIGURES

The drawing Figures depict one or more implementations in accord with the present concepts, by way of example only, not by way of limitations. In the figures, like reference numerals refer to the same or similar elements.

FIG. 1 is a cross-sectional view of a spherificated medicament according to a fi embodiment of the present invention.

FIG. 2 is top perspective cross-sectional view of a toothbrush containing a spherificated medicament according to another embodiment of the present invention.

### DESCRIPTION OF THE INVENTION

In the following detailed description, numerous specific details are set forth by way of examples in order to provide a thorough understanding of the relevant teachings. However, it should be apparent to those skilled in the art that the present teachings may be practiced without such details.

It is an aspect of the present invention to provide a spherificated form of a dental medicament composition, such as mouth rinses, mouthwashes, and dental pastes or gels. As is known by those of skill in the art, spherification is a process of shaping a liquid into spheres, such as the process described in GB 556,718, the contents of which are incorporated herein by reference in its entirety. The process generally comprises temporarily immersing a mixture containing a soluble alginate, such as sodium alginate, into a solution of calcium salt, such as calcium chloride, calcium lactate, or calcium lactate gluconate, to form a thin gelled membrane about the outer surface of the immersed composition.

Alternatively, reverse spherification may be used, which comprises temporarily immersing a mixture containing a calcium salt into an alginate solution. The alginate solution preferably further comprises a thickener or other viscosity modifiers to preferably increase the viscosity of the alginate solution. Examples of thickeners include, but are not limited to carrageenans, gellan gum, locust bean gum, xanthan gum, and mixtures thereof.

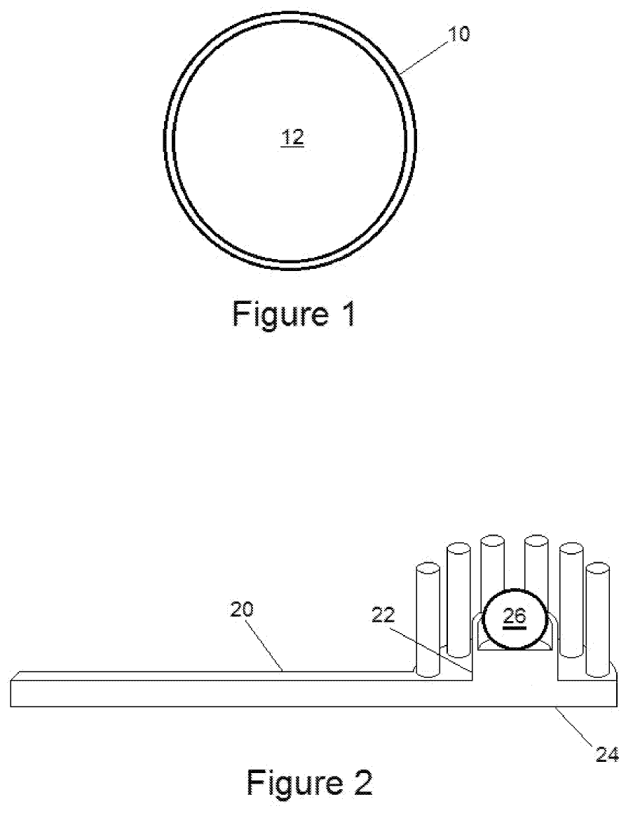

Thus, in one method according to a first embodiment of the present invention, a dental medicament composition containing a soluble alginate is immersed into a solution containing a calcium salt. The immersion step may include emulsification in which the dental medicament composition forms the discontinuous phase of the emulsion. Alternatively, the dental medicament composition may be introduced into the calcium salt solution by injecting drops of the composition via a syringe, for example. The dental medicament composition remains immersed until a membrane is formed about individual droplets of the dental medicament composition. An example of a spherificated dental medicament is illustrated in FIG. 1, which comprises a membrane 10 encapsulating a core 12 containing the dental medicament composition.

Once a sufficiently rigid membrane has formed about the droplets, the encapsulated droplets may be removed and optionally rinsed and/or subjected to some temperature and/or humidity conditioning, as well as optional surface treatments with an anti-tacking agent to prevent agglomeration of the spherificated medicament prior to packaging. Examples of anti-tacking agents include, but are not limited to, talc, silica, fumed silica, colloidal silica dioxide, calcium stearate, carnauba wax, long chain fatty alcohols and waxes, such as stearic acid and stearyl alcohol, mineral oil, paraffin, micro crystalline cellulose, glycerin, propylene glycol, and polyethylene glycol.

As explained above, the processes according to the present invention may utilize reverse spherification techniques, whereby a dental medicament containing a calcium salt may be immersed into an alginate solution.

The dental medicament compositions according to the various embodiments of the present invention, in addition to containing either a soluble alginate or calcium salt, may further include effective concentrations of active ingredients, such as fluoride or antimicrobial agents. Examples of antimicrobial agents include, but are not limited to, cetylpyridinium chloride and triclosan. Fluoride may be provided by one or more fluoride ion sources, such as stannous fluoride, sodium fluoride, potassium fluoride, sodium monofluorophosphate, sodium fluorosilicate, ammonium fluorosilicate, amine fluoride, ammonium fluoride, and combinations thereof.

Other ingredients that may be incorporated in the compositions according to the present invention include, but are not limited to, humectants, such as glycerine, sorbitol, polyethylene glycol, non-toxic alcohols, such as ethanol, thickeners or gelling agents, such as polyvinylpyrolidone, hydroxyethyl propylcellulose, hydroxybutyl methyl cellulose, hydroxypropyl methyl cellulose, hydroxyethyl cellulose, sodium carboxymethyl cellulose and the like, and surfactants, such as polyoxyethylene polyoxypropylene block polymers available under the tradename “Pluronics”, an amido betaine such as cocoamido propyl betaine and water soluble salts of higher fatty acid monoglyceride monosulfates, such as sodium salts of the monosulfated monoglycerides, or hydrogenated coconut oil fatty acids, higher alkylsulfate, such as sodium lauryl sulfate and alkyl aryl sulfonates, such as sodium dodecyl benzene sulfonate. A natural or synthetic sweetening agent, such as dextrose, levulose, saccharin, or cyclamate as well as flavors such as oils of spearmint, peppermint, wintergreen, may be included in the compositions according to the present invention. By relying on emulsion processes or droplet forming techniques, for example, in the immersion step of the above-described processes, the volume of the individual droplets of the dental medicament composition may be controlled. The spherificated medicaments according to the various embodiments of the present invention may have a diameter of about 1 to 20 mm, more preferably about 1 to 15 mm, and most preferably about 1 to 10 mm.

In one aspect, the size and dose of the final encapsulated product may be precisely controlled to provide individual single-use dosages. The individual encapsulated dental medicament composition may, for example, provide the exact volume of mouthwash recommended for either an adult or child. In another aspect, the single use dosages may be more hygienic than a toothpaste tube or mouthwash bottle, for example, because it avoids the potential for multiples users contacting the opening of the containers with different toothbrushes or their mouths. It may also avoid residual medicament building up around the outside of the opening of the container over time.

In another embodiment, the membrane of the spherificated product may encapsulate a toothpaste or gel having a relatively high concentration of water. Applying the dosage of the encapsulated medicament to a toothbrush and breaking the membrane during brushing will enable release and foaming of the toothpaste or gel without the need to wet the toothbrush. Therefore, this embodiment of the invention may provide a dosage of toothpaste or gel that may be used in a location without access to running water, such as a campsite, for example.

In yet another embodiment of the present invention, the encapsulated toothpaste or gel compositions according to the various embodiments of the present invention may also be combined with a disposable toothbrush, such as the embodiment illustrated in FIG. 2, to provide a single-use toothbrush. A single dose of the dental medicament composition may be pre-loaded in the brush section 24 of the toothbrush 20, The brush section 24 of the toothbrush 20 may comprise a receptacle 22 configured to receive one or more pellets 26 of the encapsulated toothpaste or gel. Alternatively, the pellets may be sold separately, so that a multiple-use toothbrush having a receptacle in the brush section may be repeatedly loaded prior to every use.

While particular embodiments of the present invention have been illustrated and described, it would be obvious to those skilled in the art that various other changes and modifications can be made without departing from the spirit and scope of the invention.

I claim:

1. A spherificated dental medicament comprising:
an outer membrane comprising calcium alginate; and
an internal core containing at least one of a mouth rinse, a mouthwash, a dental paste, and a dental gel.

2. The spherificated dental medicament of claim 1, wherein the internal core further comprises an antimicrobial agent.

3. The spherificated dental medicament of claim 2, wherein the antimicrobial agent is selected from the group consisting of cetylpyridinium chloride and triclosan.

4. The spherificated dental medicament of claim 1, wherein the internal core further comprises a fluoride containing compound.

5. The spherificated dental medicament of claim 4, wherein the fluoride containing compound is selected from the group consisting of stannous fluoride, sodium fluoride, potassium fluoride, sodium monofluorophosphate, sodium fluorosilicate, ammonium fluorosilicate, amine fluoride, and ammonium fluoride.

6. The spherificated dental medicament of claim 1, wherein the outer membrane has a diameter of 1 to 20 mm.

7. A toothbrush in combination with the spherificated dental medicament of claim 1.

8. The toothbrush of claim 7 further comprising a head having a plurality of bristles and a compartment containing the spherificated dental medicament.

9. A method of making a spherificated dental medicament comprising:
immersing a dental medicament composition into a solution containing a calcium salt the dental medicament composition comprising a soluble alginate and at least one of a mouth rinse, a mouthwash, a dental paste and a dental gel; and
removing the dental medicament composition from the solution following formation of a membrane around the dental medicament composition, the membrane comprising calcium alginate.

10. The method of claim 9, wherein the immersing step comprises introducing the dental medicament composition dropwise into the solution containing the calcium salt.

11. The method of claim 9, wherein the immersing step comprises forming an emulsion comprising a non-continuous phase containing the dental medicament composition and a continuous phase containing the solution containing the calcium salt.

12. A method of making a spherificated dental medicament comprising:
immersing a dental medicament composition into a solution containing a soluble alginate, the dental medicament composition comprising a calcium salt and at least one of a mouth rinse, a mouthwash, a dental paste and a dental gel; and
removing the dental medicament composition from the solution following formation of a membrane around the dental medicament composition, the membrane comprising calcium alginate.

13. The method of claim 12, wherein the immersing step comprises introducing the dental medicament composition dropwise into the solution containing the soluble alginate.

14. The method of claim 12, wherein the immersing step comprises forming an emulsion comprising a non-continuous phase containing the dental medicament composition and a continuous phase containing the solution containing the soluble alginate.
